(19) United States
(12) Patent Application Publication
(10) Pub. No.: US 2020/0114128 A1
(43) Pub. Date: Apr. 16, 2020

(54) TARGETING GUIDEWIRE

(71) Applicant: Cook Medical Technologies LLC, Bloomington, IN (US)

(72) Inventors: Blayne A. ROEDER, Bloomington, IN (US); Elizabeth A. EATON, Bloomington, IN (US)

(73) Assignee: Cook Medical Technologies LLC, Bloomington, IN (US)

(21) Appl. No.: 16/653,132

(22) Filed: Oct. 15, 2019

Related U.S. Application Data
(60) Provisional application No. 62/746,209, filed on Oct. 16, 2018.

(51) Int. Cl. A61M 25/09; A61M 25/00

(52) U.S. Cl. CPC A61M 25/09; A61M 25/0054; A61M 2025/09166; A61M 2025/09175; A61M 2025/0915; A61M 2025/09133; A61M 2025/09091

(57) ABSTRACT

The disclosure is directed to a targeting guidewire. The guidewire includes a distal portion including four portions and three transition zones between the adjacent portions. A first and a third portions have a smaller stiffness than a second and a fourth portions. The first and the third portions may also have a smaller overall outer diameters than the second and the fourth portions. The three transition zones comprises marking elements so as to delineate their locations.

### RELATED APPLICATION

This application claims priority to Provisional Patent Application No. 62/746,209, filed on Oct. 16, 2018, which is incorporated by reference in its entirety.

### BACKGROUND

### 1. Technical Field

The present disclosure relates to guidewires. In particular, the present disclosure relates to targeting guidewires.

### 2. Background Information

As endovascular aortic intervention patient demographics change (more patients being treated endovascularly, at younger ages, with more complex disease, living longer, and requiring more re-interventions), more and more medical procedures necessitate significant catheter and wire manipulation to reach target vessels through fenestrations and branches.

Navigating from a graft lumen through a fenestration and into a target vessel involves taking a sharp turn—for example, around 90° from an aorta into a renal artery. The accessory devices are being controlled at the proximal end, outside the patient's body, by the operator, and the forces they exert on these are all being generated not in the same plane as the target plane. The controlled motions at the proximal end need to be translated through the device, and result in the desired movement of the distal end inside the vessel. This is complicated by the potential movements of the fenestration and the target artery, as well as anatomical and pathological conditions in vivo.

Guidewires are often used to facilitate intravascular access to gain access to target vessels for treatment. During these procedures, the availability of the right guidewire for the task is essential.

### BRIEF SUMMARY

The present disclosure is directed to a guidewire. The guidewire includes a distal section, a proximal section, and a middle section between the distal section and the proximal section. A first portion of a first stiffness disposed at a distal end of the distal section, the first portion having a first overall outer diameter. A second portion of a second stiffness having a second overall outer diameter and disposed adjacent to the first portion. A third portion of a third stiffness having a third overall outer diameter and disposed adjacent to the second portion. A fourth portion of a fourth stiffness disposed adjacent to the third portion and configured to connect to the middle section, the fourth portion having a fourth overall outer diameter.

A first transition zone is disposed between the first portion and the second portion, wherein a first end of the first transition zone is adjacent to the first portion and has a same overall outer diameter as the first portion. A second end of the first transition zone is adjacent to the second portion and has a same overall outer diameter as the second portion. A second transition zone is disposed between the second portion and the third portion, wherein a first end of the second transition zone is adjacent to the second portion and has a same overall outer diameter as the second portion. A second end of the second transition zone is adjacent to the third portion and has a same overall outer diameter as the third portion. A third transition zone is disposed between the third portion and the fourth portion, wherein a first end of the third transition zone is adjacent to the third portion and has a same overall outer diameter as the third portion. A second end of the third transition zone is adjacent to the fourth portion and has a same overall outer diameter as the fourth portion. Furthermore, the first overall outer diameter and the third overall outer diameter are smaller than the second overall outer diameter and the fourth overall outer diameter, the first stiffness and third stiffness are less stiff than the second stiffness and fourth stiffness, and the first transition zone, the second transition zone, and the third transition zone comprise marking elements so as to delineate their locations.

The present disclosure also describes a system for guiding a device into a target vessel. The system includes a guidewire, and the guidewire includes a distal section, a proximal section, and a middle section between the distal section and the proximal section. A first portion of a first stiffness disposed at a distal end of the distal section, the first portion having a first overall outer diameter. A second portion of a second stiffness having a second overall outer diameter and disposed adjacent to the first portion. A third portion of a third stiffness having a third overall outer diameter and disposed adjacent to the second portion. A fourth portion of a fourth stiffness disposed adjacent to the third portion and configured to connect to the middle section, the fourth portion having a fourth overall outer diameter. A first transition zone is disposed between the first portion and the second portion, wherein a first end of the first transition zone is adjacent to the first portion and has a same overall outer diameter as the first portion. A second end of the first transition zone is adjacent to the second portion and has a same overall outer diameter as the second portion. A second transition zone is disposed between the second portion and the third portion, wherein a first end of the second transition zone is adjacent to the second portion and has a same overall outer diameter as the second portion. A second end of the second transition zone is adjacent to the third portion and has a same overall outer diameter as the third portion. A third transition zone is disposed between the third portion and the fourth portion, wherein a first end of the third transition zone is adjacent to the third portion and has a same overall outer diameter as the third portion. A second end of the third transition zone is adjacent to the fourth portion and has a same overall outer diameter as the fourth portion. Furthermore, the first overall outer diameter and the third overall outer diameter are smaller than the second overall outer diameter and the fourth overall outer diameter, the first stiffness and third stiffness are smaller than the second stiffness and fourth stiffness, and the first transition zone, the second transition zone, and the third transition zone comprise marking elements so as to delineate their locations.

### BRIEF DESCRIPTION OF THE DRAWINGS

FIG. 1 is a schematic diagram of a targeting guidewire.

FIG. 2 is a schematic diagram of a distal section of a targeting guidewire in one embodiment.

FIG. 3A is a schematic diagram of a curved shape of a first portion in one embodiment.

FIG. 3B is a schematic diagram of a standard J-shape of a first portion in one embodiment.

FIG. 3C is a schematic diagram of a tightened J-shape of a first portion in one embodiment.

FIG. 4A is a schematic diagram of a portion of a guidewire inside a sheath in one embodiment.

FIG. 4B is a schematic diagram of another portion of the guidewire inside the sheath as in FIG. 4A.

FIG. 5 is an illustration of a targeting wire being manipulated into a renal artery of a body.

### DETAILED DESCRIPTION OF THE DISCLOSURE

The present disclosure will now be described in detail hereinafter with reference to the accompanied drawings, which form a part of the present disclosure, and which show, by way of illustration, specific examples of embodiments. Please note that the disclosure may, however, be embodied in a variety of different forms and, therefore, the covered or claimed subject matter is intended to be construed as not being limited to any of the embodiments to be set forth below. Please also note that the disclosure may be embodied as methods, devices, components, or systems. Accordingly, embodiments of the disclosure may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” or “in other embodiments” as used herein does not necessarily refer to a different embodiment. It is intended, for example, that claimed subject matter includes combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

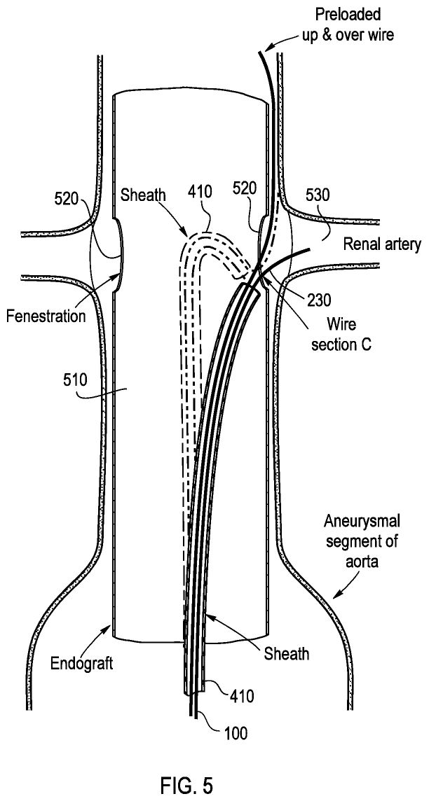

FIG. 5 shows a schematic diagram of one embodiment, where a targeting guidewire 100 is manipulated through a graft 510 situated inside an aorta, out through a fenestration 520, and then into a renal artery 530. As illustrated in FIG. 5, the renal artery 530 is about 90 degree relatively to the graft 510. With the targeting guidewire 100 and the sheath 410, the targeting guidewire may be fed through and into the renal artery safely and easily.

FIG. 1 shows a schematic diagram of a targeting guidewire 100. The guidewire includes a distal section 110, a middle section 120, and a proximal section 130. Herein, “distal” may be used to refer to a portion of a device further away from a health care provider handling the device when in use. “Proximal” may be used to refer to a portion of a device closer to a health care provider handling the device when in use. The three sections may have various length. In one embodiment, the distal section 110 may be used to enter a target vessel, and the proximal section 130 may be closer to a physician or outside of a patient's body. In some embodiments, the middle section may be much longer than the distal and proximal sections. The distal section and proximal section may have the same length. In some embodiments, the distal section and proximal section may have different lengths.

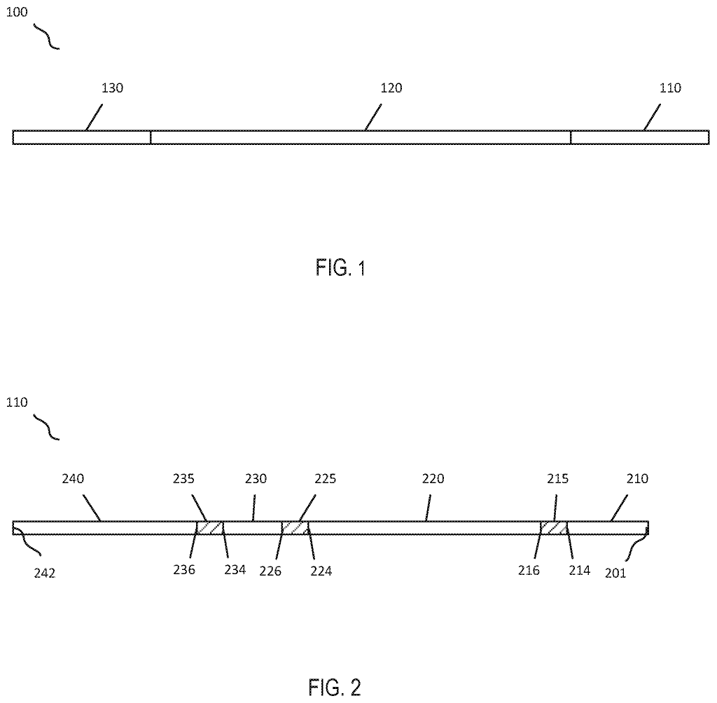

FIG. 2 shows a schematic diagram of a distal section 110 in some embodiments. The distal section 110 may include a distal end 201 and a proximal end 242. The distal section 110 may also include four portions: a first portion 210, a second portion 220, a third portion 230, and a fourth portion 240. The four portions are connected by corresponding transition zones between the adjacent portions. A first transition zone 215 is between the first portion 210 and the second portion 220. A second transition zone 225 is between the second portion 220 and the third portion 230. A third transition zone 235 is between the third portion 230 and the fourth portion 240. In some embodiments, the distal section 110 may include two or three portions. In other embodiments, the distal section 110 may include more than four portions, for example and not limited to, ten portions.

Several portions of the portions of the distal section 110 may have different stiffness or same stiffness; may have the same length or different length; or may have the same overall outer diameter or different overall outer diameter. In one embodiment, one portion of the distal section may have a uniform overall outer diameter along the length of the portion. In other embodiments, one portion of the distal section may have a non-uniform overall outer diameter along the length of the portion.

One or more transition zones between adjacent portions of the distal section may have different stiffness or same stiffness; may have the same length or different length; or may have the same overall outer diameter or different overall outer diameter. In one embodiment, one transition zone may have a non-uniform overall outer diameter along the length of the portion.

The portions of the guidwire may be made of materials, which include and are not limited to stainless steel, nitinol (nickel titanium alloy), platinum, and gold. The sections of the guidewire 100 may be coated with one or more layers. In one embodiment, the guidewire 100 may be coated for improved lubricity. The coating may be silicone, polytetrafluoroethylene, other hydrophilic coating, or other lubricious coating.

The transition zones between adjacent portions may include marking elements so that the positions of the transition zones may be imaged with a corresponding imaging method when the guidewire is disposed inside a body of a patient. The marking elements may include but are not limited to radiodensity-dissimilar metals, etched marks, and radiopaque bands. The marking elements may be provided in a variety of ways. In one embodiment, the marking elements may be formed as integral parts of one or more transitional zones. In another embodiment, the marking elements may be separately attached to one or more transitional zones, for example and not limited to, by bonding and by coating.

Materials, for example metals, may show up more or less brightly due to their more or less radiopaque under x-ray or fluoroscopy. When materials have dissimilar radiodensity, which may be an inherent property of the materials but may also be a function of thickness/geometry, shape/arrangement, etc, they will appear differently under radiation, allowing a health care provider to distinguish between marked sections of a device. Two identical metal slugs of the same size and shape, implanted in a patient (or a phantom model) at the same location will appear identical. However, if one slug is made of different radiodensity material but is in other ways same to the other slug, one slug may appear considerably less bright or visible than the other slug. For example, when one slug is made of stainless steel while the other slug is made of platinum, the one slug made of the stainless steel may appear less bright/visible than the other slug made of platinum, because platinum is more radiopaque than stainless steel as a material. In one implementation, radiodensity, radiodense, and radiopaque are frequently used interchangeably—the antonym would be radiolucent.

As shown in FIG. 2, the first portion 210 may have a first stiffness and a first overall outer diameter. The first overall outer diameter may be any value between about 0.01 inch and about 0.03 inch, for example and not limited to, 0.014 inch, 0.018 inch, and 0.025 inch. The first stiffness is between about 0.005 Newton and about 0.1 Newton. A length of the first portion 210 may be any value between about 1 cm and about 2 cm. The term “about” is specifically defined herein and in the whole present disclosure to include the reference value and means a range of values plus or minus 5 or 10% of the reference value.

The second portion 220 may have a second stiffness and a second overall outer diameter. The second overall outer diameter may be any value between about 0.01 inch and about 0.05 inch, for example and not limited to, 0.018 inch, 0.025 inch, and 0.035 inch. The second stiffness is larger than about 0.5 Newton. A length of the second portion 220 may be any value between about 15 cm and about 20 cm.

The third portion 230 may have a third stiffness and a third overall outer diameter. The third overall outer diameter may be any value between about 0.01 inch and about 0.03 inch, for example and not limited to, 0.014 inch, 0.018 inch, and 0.025 inch. The third stiffness is between about 0.01 Newton and about 0.1 Newton. A length of the third portion 230 may be any value between about 1 cm and about 5 cm.

The fourth portion 240 may have a fourth stiffness and a fourth overall outer diameter. The fourth overall outer diameter may be any value between about 0.01 inch and about 0.05 inch, for example and not limited to, 0.018 inch, 0.025 inch, and 0.035 inch. The fourth stiffness is larger than about 0.5 Newton. A length of the fourth portion 220 may be any value larger than about 1 cm. The proximal end 242 is connected to the middle section 120 of the guidewire 100.

The first transition zone 215 has a first end 214 and a second end 216. The first end 214 of the first transition zone is adjacent to the first portion 210. The outer diameter of the first end 214 is the same as the outer diameter of the first portion 210, so that a smooth transition exists between the first portion 210 and the first transition zone 215. The second end 216 of the first transition zone is adjacent to the second portion 220. The outer diameter of the second end 216 is the same as the outer diameter of the second portion 220, so that a smooth transition exists between the first transition zone 215 and the second portion 220. When the outer diameter of the first end 214 and second end 216 are different, the first transition zone 215 may change its outer diameter gradually from its first end 214 to its second end 216. In one implementation, the outer diameter of the first transition zone 215 may change linearly from its first end 214 to its second end 216, so that an angel of a taper of the outer surface of the first transition zone 215 is between 2 degrees and 60 degrees, inclusive. In another implementation, the outer diameter of the first transition zone 215 may change nonlinearly from its first end 214 to its second end 216

The second transition zone 225 has a first end 224 and a second end 226. The first end 224 of the second transition zone is adjacent to the second portion 220. The outer diameter of the first end 224 is the same as the outer diameter of the second portion 220, so that a smooth transition exists between the second portion 220 and the second transition zone 225. The second end 226 of the second transition zone is adjacent to the third portion 230. The outer diameter of the second end 226 is the same as the outer diameter of the third portion 230, so that a smooth transition exists between the second transition zone 225 and the third portion 230. When the outer diameter of the first end 224 and second end 226 are different, the second transition zone 225 may change its outer diameter gradually from its first end 224 to its second end 226, similar to the description of the transition zone 215.

The third transition zone 235 has a first end 234 and a second end 236. The first end 234 of the third transition zone is adjacent to the third portion 230. The outer diameter of the first end 234 is the same as the outer diameter of the third portion 230, so that a smooth transition exists between the third portion 230 and the third transition zone 235. The second end 236 of the third transition zone is adjacent to the fourth portion 240. The outer diameter of the second end 236 is the same as the outer diameter of the fourth portion 240, so that a smooth transition exists between the third transition zone 235 and the fourth portion 240. When the outer diameter of the first end 234 and second end 236 are different, the third transition zone 235 may change its outer diameter gradually from its first end 234 to its second end 236, similar to the description of the transition zone 215.

In one embodiment, the end 242 of the fourth portion has the same outer diameter as the middle section 120, so that a smooth transition exists between the fourth portion 240 and the middle section 120.

In another embodiment, the first stiffness and the third stiffness may be smaller than the second stiffness and fourth stiffness. For example and not limited to, the first stiffness is 0.05 Newton; the third stiffness is either the same as the first stiffness or slightly different at 0.08 Newton; the third stiffness is about 1 Newton; and the fourth stiffness is either the same as the third stiffness or slightly different at 0.8 Newton.

In some further embodiments, the first overall outer diameter and the third overall outer diameter are smaller than the second overall outer diameter and the fourth overall outer diameter. For example and not limited to, the first overall outer diameter is 0.018 inch; the third overall outer diameter is either the same as the first overall outer diameter or different at 0.014 inch; the third overall outer diameter is about 0.025 inch; and the fourth overall outer diameter is either the same as the third overall outer diameter or different at 0.035 inch.

When the first stiffness and the third stiffness are smaller than the second stiffness and fourth stiffness, the first overall outer diameter, the second overall outer diameter, the third overall outer diameter, and the fourth overall outer diameter may be about the same. The term “about” is specifically defined herein and in the whole present disclosure to include the reference value and means a range of values plus or minus 5 or 10% of the reference value.

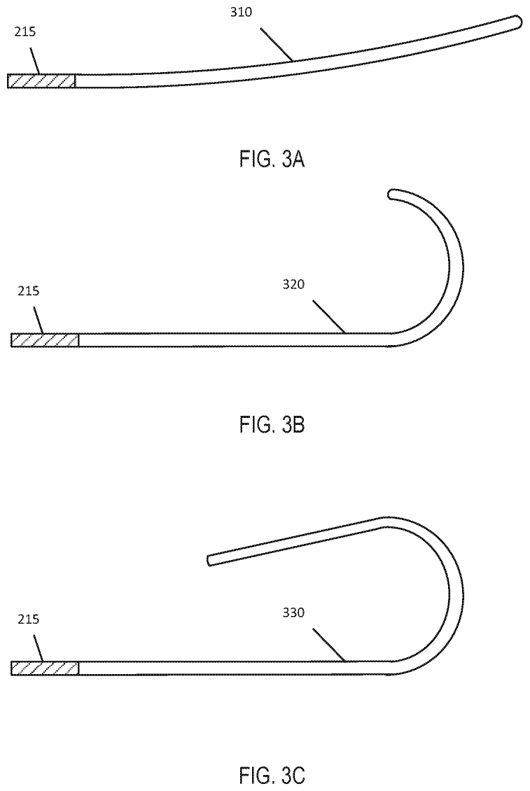

To provide maximum protection to target vessels during intravascular access by the targeting guidewire, for example, to protect the aorta and renal artery during the procedure in FIG. 5, the first portion of the distal section of the targeting guidewire may have a curved shape. As in FIG. 3A, the first portion 310 has a curved shape. As in FIG. 3B, the first portion 310 has a standard J-shape. As in FIG. 3C, the first portion 310 has a tightened J-shape. The J-shape of the first portion protects the vessel and delicate tissues as the guidewire is advanced into the vessel. A curved and the standard or tightened J-shape is more easily deflected and prevents the distal end of the guidewire from exerting a dangerous amount of force against the vessel wall. Curved distal ends on guide wires can also be used to broaden the directional options when advancing a guide wire inside of a catheter.

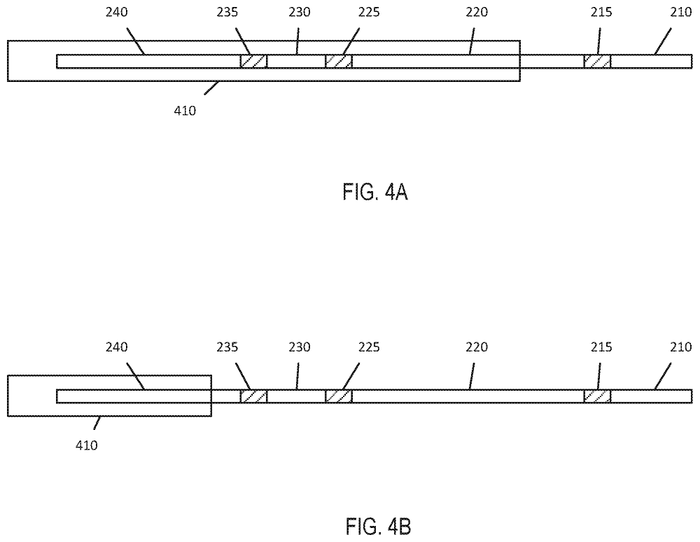

As illustrated in FIGS. 4A and 4B, a targeting guidewire may be used with a sheath 410 to provide a user with a high level of control and manipulability. The inner diameter of the sheath 410 may be slightly larger than the largest overall outer diameter of the guidewire 100, so that the guidewire 100 can slide smoothly inside the sheath without kinking inside the sheath 410.

In FIG. 4A, the sheath 410 encloses a portion of the second portion 220, the third portion 230, and the fourth portion 240. By retaining a portion of the second portion 220 inside the sheath 410 as in FIG. 4A, the guidewire may be manipulated much the similar way as a regular guidewire.

In FIG. 4B, a sheath 410 encloses a portion of the fourth portion 240, and the first, second, and third portions are outside the sheath. In some embodiments, the sheath 410 may encloses a portion of the third portion 230 and the fourth portion 240. When the stiffness of the third portion 230 is smaller than the second portion 220, the second portion 220 may be manipulated more independently, so that the first or second portions may be easily manipulated into the target vessel.

In some procedures, a medical provider, for example, a physician, may want to introduce the guidewire into a target vessel having a sharp angle relatively to the main vessel, for example, the renal artery 530 in FIG. 5. The physician may adjust the relative position of the sheath 410 and a targeting guidewire 100 so that a portion of the second portion 220 is inside the sheath. In this configuration, the physician can target a renal artery as usual with a normal guidewire. Further, when the first portion 210 reaches the renal artery, the physician may advance the targeting guidewire relative to the sheath so that a significant portion of the third portion 230 is out of the sheath. The physician may then anchor the guidewire and advance the sheath relative to the guidewire, so that the distal end 110 of the targeting guidewire may be more easily manipulated as a curve into the renal artery.

Once the sheath is in place inside the target vessel, the guidewire may potentially be withdrawn out of and/or replaced by a more standard guide wire in the sheath, and the physician can continue with additional aspects of treatment, for example, bringing in balloons or stents directly to the target vessel or injecting contrast directly into the target vessel for imaging purposes.

The present disclosure also describes a system for guiding a device or prosthesis. The system may include a guidewire as described in the above embodiments. The device may be a catheter.

While the particular disclosure has been described with reference to illustrative embodiments, this description is not meant to be limiting. Various modifications of the illustrative embodiments and additional embodiments of the disclosure will be apparent to one of ordinary skill in the art from this description. Those skilled in the art will readily recognize that these and various other modifications can be made to the exemplary embodiments, illustrated and described herein, without departing from the spirit and scope of the present disclosure. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. A guidewire, comprising:
a distal section, a proximal section, and a middle section between the distal section and the proximal section;
a first portion of a first stiffness disposed at a distal end of the distal section, the first portion having a first overall outer diameter;
a second portion of a second stiffness having a second overall outer diameter and disposed adjacent to the first portion;
a third portion of a third stiffness having a third overall outer diameter and disposed adjacent to the second portion;
a fourth portion of a fourth stiffness disposed adjacent to the third portion and configured to connect to the middle section, the fourth portion having a fourth overall outer diameter;
a first transition zone disposed between the first portion and the second portion, wherein a first end of the first transition zone is adjacent to the first portion and has a same overall outer diameter as the first portion, and a second end of the first transition zone is adjacent to the second portion and has a same overall outer diameter as the second portion;
a second transition zone disposed between the second portion and the third portion, wherein a first end of the second transition zone is adjacent to the second portion and has a same overall outer diameter as the second portion, and a second end of the second transition zone is adjacent to the third portion and has a same overall outer diameter as the third portion;
a third transition zone disposed between the third portion and the fourth portion, wherein a first end of the third transition zone is adjacent to the third portion and has a same overall outer diameter as the third portion, and a second end of the third transition zone is adjacent to the fourth portion and has a same overall outer diameter as the fourth portion; and
wherein the first overall outer diameter and the third overall outer diameter are smaller than the second overall outer diameter and the fourth overall outer diameter,
the first stiffness and third stiffness are less stiff than the second stiffness and fourth stiffness, and
the first transition zone, the second transition zone, and the third transition zone comprise marking elements so as to delineate their locations.

2. The guidewire of claim 1, wherein the first overall outer diameter is within a range of 0.014 inch and 0.025 inch, inclusive; and
the third overall outer diameter is 0.014 inch and 0.025 inch, inclusive.

3. The guidewire of claim 2, wherein the first overall outer diameter is 0.018 inch.

4. The guidewire of claim 2, wherein the third overall outer diameter is 0.018 inch.

5. The guidewire of claim 1, wherein the second overall outer diameter is 0.018 inch and 0.035 inch, inclusive; and
the fourth overall outer diameter is 0.018 inch and 0.035 inch, inclusive.

6. The guidewire of claim 5, wherein the second overall outer diameter 0.025 inch.

7. The guidewire of claim 5, wherein the fourth overall outer diameter is 0.025 inch.

8. The guidewire of claim 1, wherein the first overall outer diameter is same as the third overall outer diameter; and
the second overall outer diameter is same as the fourth overall outer diameter.

9. The guidewire of claim 1, wherein the first stiffness is between about 0.005 Newton and about 0.1 Newton;
the third stiffness is between about 0.01 Newton and about 0.1 Newton.

10. The guidewire of claim 1, wherein the second stiffness is greater than about 0.5 Newton; and
the fourth stiffness is greater than about 0.5 Newton.

11. The guidewire of claim 10, wherein the fourth stiffness is greater than the second stiffness.

12. The guidewire of claim 1, wherein an overall outer diameter of the first transition zone changes gradually from the first end to the second end of the first transition zone;
an overall outer diameter of the second transition zone changes gradually from the first end to the second end of the second transition zone; and
an overall outer diameter of the third transition zone changes gradually from the first end to the second end of the third transition zone.

13. The guidewire of claim 1, wherein the marking elements are selected from the group consisting of a radiodensity-dissimilar metal, an etched mark, and a radiopaque band.

14. The guidewire of claim 1, wherein a length of the first portion is between about 1 cm and about 2 cm,
a length of the second portion is between about 15 cm and about 20 cm, and
a length of the third portion is between about 1 cm and about 5 cm.

15. A system for guiding a device into a target vessel, the system comprising a guidewire, wherein the guidewire comprises:
a distal section, a proximal section, and a middle section between the distal section and the proximal section;
a first portion of a first stiffness disposed at a distal end of the distal section, the first portion having a first overall outer diameter;
a second portion of a second stiffness having a second overall outer diameter and disposed adjacent to the first portion;
a third portion of a third stiffness having a third overall outer diameter and disposed adjacent to the second portion;
a fourth portion of a fourth stiffness disposed adjacent to the third portion and configured to connect to the middle section, the fourth portion having a fourth overall outer diameter;
a first transition zone disposed between the first portion and the second portion, wherein a first end of the first transition zone is adjacent to the first portion and has a same overall outer diameter as the first portion, and a second end of the first transition zone is adjacent to the second portion and has a same overall outer diameter as the second portion;
a second transition zone disposed between the second portion and the third portion, wherein a first end of the second transition zone is adjacent to the second portion and has a same overall outer diameter as the second portion, and a second end of the second transition zone is adjacent to the third portion and has a same overall outer diameter as the third portion;
a third transition zone disposed between the third portion and the fourth portion, wherein a first end of the third transition zone is adjacent to the third portion and has a same overall outer diameter as the third portion, and a second end of the third transition zone is adjacent to the fourth portion and has a same overall outer diameter as the fourth portion; and
wherein the first overall outer diameter and the third overall outer diameter are smaller than the second overall outer diameter and the fourth overall outer diameter,
the first stiffness and third stiffness are less stiff than the second stiffness and fourth stiffness, and
the first transition zone, the second transition zone, and the third transition zone comprise marking elements so as to delineate their locations.
